(19) United States
(12) Patent Application Publication
(10) Pub. No.: US 2025/0064867 A1
(43) Pub. Date: Feb. 27, 2025

(54) METHOD FOR PREVENTING OR TREATING MUSCLE DISEASE

(71) Applicant: KOREA RESEARCH INSTITUTE OF BIOSCIENCE AND BIOTECHNOLOGY, Daejeon (KR)

(72) Inventors: Chul-Ho LEE, Daejeon (KR); Byoung-Chan KIM, Daejeon (KR); Yong-Hoon KIM, Daejeon (KR); Jung-Ran NOH, Daejeon (KR); Jae-Hoon KIM, Daejeon (KR); Kyoung-Shim KIM, Daejeon (KR); Dong-Hee CHOI, Daejeon (KR); Young-Keun CHOI, Daejeon (KR); Dong-Ho CHANG, Daejeon (KR); Haiyoung JUNG, Daejeon (KR); Jung Hwan HWANG, Daejeon (KR)

(73) Assignee: KOREA RESEARCH INSTITUTE OF BIOSCIENCE AND BIOTECHNOLOGY, Daejeon (KR)

(21) Appl. No.: 18/889,046

(22) Filed: Sep. 18, 2024

Related U.S. Application Data
(63) Continuation-in-part of application No. 17/512,813, filed on Oct. 28, 2021, now abandoned.
Application No. 17/512,813 is a continuation-in-part of application No. PCT/KR2019/012024, filed on Sep. 18, 2019.

(30) Foreign Application Priority Data
Oct. 1, 2018 (KR) 10-2018-0117123

(51) Int. Cl. A61K 35/741; A61K 35/00; A61K 45/06; A61P 21/00

(52) U.S. Cl. CPC A61K 35/741; A61K 45/06; A61P 21/00; A61K 2035/115

(57) ABSTRACT

The present invention relates to a method for improving muscle strength, and/or muscle mass by administering a composition containing as an active ingredient at least one selected from the group consisting of Akkermansia muciniphila cells, a culture thereof and a lysate thereof, and a method for preventing or treating muscle diseases where muscle strength and/or mass is weakened by sarcopenia, cachexia or muscle wasting.

### CROSS-REFERENCE TO RELATED APPLICATION

This application is a continuation in part of U.S. patent application Ser. No. 17/512,813 filed on Oct. 28, 2021, which is a continuation in part of PCT/KR2019/012024 filed on Sep. 18, 2019, which claims priority to and the benefit of Korean Patent Application No. 10-2018-0117123, filed on Oct. 1, 2018, the disclosures of which are incorporated herein by reference in their entirety.

Incorporation-by-Reference of Material Submitted by BY U.S.P.T.O. EFS-WEB The instant application contains a Sequence Listing, which is being submitted in computer readable form via the United States Patent and Trademark Office eFS-WEB system and which is hereby incorporated by reference in its entirety for all purposes. The XML file submitted herewith contains a 7.78 KB file (NewApp_J161410004CIP_SeqListing), which was created on Sep. 18, 2024.

### BACKGROUND

### Technical Field

The present invention relates to a method for preventing or treating a muscle disease, and a method for increasing muscle strength and/or muscle mass, comprising administering a composition containing as an active ingredient at least one selected from the group consisting of Akkermansia muciniphila cells, a culture thereof and a lysate thereof.

### Background Art

Sarcopenia is a medical condition characterized by the progressive loss of muscle mass, strength, and function. It can significantly impact an individual's mobility, balance, and overall quality of life. The pathology of sarcopenia is complex, but it is known that muscle atrophy plays a significant role in development of sarcopenia [Medicine and Science in Sports and Exercise, 26(4):432-439, 1994/Physiological Reviews, 99: 427-511, 2019], It is also known that muscle atrophy is a common feature of sarcopenia and cachexia [Gerontology, 60(4): 294-305, 2014]. Cachexia is a complex metabolic syndrome characterized by severe weight loss, muscle wasting, and a significant reduction in fat mass, often accompanied by weakness and fatigue. Loss of muscle mass, strength and functions often happens in significant weight loss, for example, by medication such as GLP-1 (glucagon like peptide-1) agonist. It is known that there is a potential risk of muscle atrophy is such a significant weight loss in the absence of adequate dietary protein and physical activity [Journal of Cachexia, Sarcopenia and Muscle 10: 903-918, 2019].

Muscle atrophy refers to weakening and degeneration of muscles caused by gradual decrease of muscle mass (Cell, 119(7): 907-910, 2004). Muscle atrophy is facilitated by immobility, oxidative stress and chronic inflammation, and weakens muscular functions and motor ability (Clinical Nutrition, 26(5): 524-534, 2007).

The most important factor that determines muscular functions is muscle mass, which is maintained by the balance of protein synthesis and degradation. Muscle atrophy occurs when protein degradation exceeds protein synthesis (The International Journal of Biochemistry and Cell Biology, 37(10): 1985-1996, 2005). Muscle size is controlled by the intracellular signaling pathways that lead to anabolism or catabolism in muscles. When signaling for the synthesis of muscle proteins exceeds that for the degradation of muscle proteins, muscle protein synthesis is increased, resulting in increased muscle size (hypertrophy) or increased number of muscle fibers (hyperplasia) due to increased muscle proteins (The Korea Journal of Sports Science, 20(3): 1551-1561, 2011).

Korean Patent Publication No. 2015-0093711 discloses “use of Akkermansia for treating a metabolic disorder”, but effect of increasing muscle strength and/or muscle mass of Akkermansia muciniphila have not been known.

### DISCLOSURE

### Technical Problem

The present inventors made intensive research efforts to prevent or treat muscle diseases such as sarcopenia, cachexia or muscle wasting where muscle atrophy plays a role in pathology, by using a substance non-toxic to the human body and capable of effectively increasing myogenesis and also decreasing muscle atrophy in the means of, for example, increasing mRNA expression levels of Follistatin and/or reducing mRNA expression levels of Atrogin-1 in muscle cells, and as a result, identified that Akkermansia muciniphila showed effects of preventing or treating muscle diseases such as sarcopenia, cachexia and muscle wasting by weight loss, when administered to animal models, thereby completing the present invention.

### Technical Solution

An objective of the present invention is to provide a method for preventing or treating muscle diseases where muscle atrophy plays a role in pathology in a subject in need thereof, the method comprising administering to the subject a composition comprising as an active ingredient at least one selected from the group consisting of Akkermansia muciniphila cells, a culture thereof and a lysate thereof, wherein the muscle disease is selected from the group consisting of sarcopenia, cachexia and muscle wasting by significant weight loss by medication such as GLP-1 agonists.

Another objective of the present invention is to provide a method for increasing muscle strength and/or muscle mass in a subject in need thereof, the method comprising administering to the subject a composition comprising as an active ingredient at least one selected from the group consisting of Akkermansia muciniphila cells, a culture thereof and a lysate thereof.

The compositions comprising as an active ingredient at least one selected from the group consisting of Akkermansia muciniphila cells, a culture thereof and a lysate thereof have effects of increasing myogenesis, increasing mRNA expression levels of Follistatin and/or reducing mRNA expression levels of Atrogin-1 in muscle cells, increasing muscle strength and/or muscle mass, and thus can effectively prevent and treat various muscle diseases such as sarcopenia, cachexia or muscle wasting by significant weight loss.

### DESCRIPTION

### Brief Description of Drawings

In the drawing of the present invention, Vehicle represents a control group, AK represents a live Akkermansia strain administration group, and AK-P represents a dead Akkermansia strain administration group.

FIG. 1 shows the qRT-PCR comparison in the mRNA expression levels of myogenin (Myog) and myosin heavy chain (MyHC) when C2C12 skeletal muscle myoblasts were treated with live or dead cells of Akkermansia strain.

FIG. 2 shows the qRT-PCR comparison in the mRNA expression levels of Follistatin when C2C12 skeletal muscle myoblasts were treated with dead cells of Akkermansia strain.

FIG. 3 shows the qRT-PCR comparison in the mRNA expression levels of Atrogin-1 when C2C12 skeletal muscle myoblasts were treated with dead cells of Akkermansia strain.

FIG. 4 shows the qRT-PCR comparison in the mRNA expression levels of MuRF1 when C2C12 skeletal muscle myoblasts were treated with dead cells of Akkermansia strain.

FIG. 5 shows the DEXA images when dexamethasone-induced muscle atrophy animal model was treated with dead cells of Akkermansia strain.

FIG. 6 shows the histological feature of muscles when dexamethasone-induced muscle atrophy animal model was treated with dead cells of Akkermansia strain.

FIG. 7 shows the size of the tibialis anterior (TA) muscles when dexamethasone-induced muscle atrophy animal model was treated with dead cells of Akkermansia strain.

FIG. 8 shows the percentage of the tibialis anterior (TA) muscles size when dexamethasone-induced muscle atrophy animal model was treated with dead cells of Akkermansia strain.

FIG. 9 shows the measurement of muscle strength of mice administered with the live or dead cells of Akkermansia strain, by using a grip strength meter.

FIG. 10 shows the muscle weight relative to body weight of aged mice administered with the live or dead cells of Akkermansia strain.

FIG. 11 identifies the size of muscle fibers of mice administered with the live or dead cells of Akkermansia strain and shows immunostaining images for laminin.

FIG. 12 shows the number of muscle fibers according to the size of the tibialis anterior (TA) muscle.

FIG. 13 shows the mean size of all of the tibialis anterior (TA) muscle fibers.

### DETAILED DESCRIPTION OF THE INVENTION

The present invention will be specifically described as follows. Each description and exemplary embodiment disclosed in this invention may also be applied to other descriptions and exemplary embodiments. That is, all combinations of various elements disclosed in this invention fall within the scope of the present invention. In addition, the scope of the present invention is not limited by the specific description below.

Also, a person skilled in the art could recognize or identify numerous equivalents with respect to certain aspects of the present invention only by routine experiments. Furthermore, such equivalents are intended to be encompassed by the present invention.

In accordance with an aspect of the present invention to attain the object, there is provided a method for preventing or treating a muscle disease in a subject in need thereof, the method comprising administering to the subject a composition comprising as an active ingredient at least one selected from the group consisting of Akkermansia muciniphila cells, a culture thereof and a lysate thereof, wherein the muscle disease is selected from the group consisting of muscle atrophy, sarcopenia, cachexia and muscle wasting.

And, to attain the other object, there is provided a method for increasing muscle strength and/or muscle mass in a subject in need thereof, the method comprising administering to the subject a composition comprising as an active ingredient at least one selected from the group consisting of Akkermansia muciniphila cells, a culture thereof and a lysate thereof.

“Akkermansia muciniphila” of the present invention is a gram-negative, strictly anaerobic, non-motile, non-spore-forming, oval-shaped bacterium. The Akkermansia muciniphila bacteria use mucin as sole source of carbon and nitrogen thereof, and are known to inhabit the gastrointestinal tract of various animals including humans.

“Akkermansia muciniphila” of the present invention is preferably cultured in a glucose-free medium, which comprises lactose, N-acetylhexosamine, L-threonine, cobalamin, tryptone and yeast extract. The glucose-free medium comprises lactose as a carbon source, but not glucose. Preferably, lactose is a sole carbon source in the glucose-free medium. More preferably, the glucose-free medium comprises 0.2-1 g/L of L-cysteine, 0.5-5 g of N-acetylhexosamine, 1-5 g/L of L-threonine, 5-20 g/L of yeast extract, 10-20 g/L of lactose, 10-30 g/L of tryptone and 0.01-1 mg/L of cobalamin. The glucose-free medium may further comprise 0.2-0.8 g/L of monopotassium phosphate, 0.3-0.9 g/L of disodium phosphate, 0.1-0.6 g/L of ammonium chloride, 0.05-0.4 g/L of magnesium chloride, 0.05-0.4 g/L of calcium chloride and 2-8 g of sodium bicarbonate.

Specifically, Akkermansia muciniphila of the present invention may be strains deposited at the American Type Culture Collection under accession number ATCC BAA-835 and at the German Collection of Microorganisms and Cell Cultures under accession number DSM 22959, preferably strains deposited at the Korean Culture Center of Microorganisms under accession number KCCM12424P, but could include any strain without limitation so long as the strain has an effect of increasing muscle strength and/or muscle mass. In addition, cells of the Akkermansia muciniphila strain, a culture of the strain and a lysate of the strain may also be included in the scope of the present invention.

The Akkermansia muciniphila of the present invention includes both live and dead cell forms. For example, the Akkermansia muciniphila of the present invention may be used in the form of live cells, dead cells, or a mixture of live and dead cells. For example, Akkermansia muciniphila may exist in a dried, lyophilized, or heated form. However, Akkermansia muciniphila may be used in the form suitable for inclusion in various compositions, without the limitation to the above-described examples. Preferably, Akkermansia muciniphila is dead cells, which are pasteurized and then freeze-dried. More preferably, the composition of the present invention comprises Akkermansia muciniphila dead cells and carrier, wherein a content of Akkermansia muciniphila dead cells in the composition, is 1×10⁵−1×10¹⁵ cell/g, preferably 1×10⁸−1×10¹³ cell/g, more preferably 1×10¹⁰−1×10¹² cell/g.

The term “muscle disease” of the present invention refers to a disease selected from the group consisting of, but not limited to, muscle atrophy, sarcopenia, cachexia, muscle wasting, and combinations thereof.

The muscle wasting may occur due to genetic factors, acquired factors and other factors. The acquired factors may be side-effect of drug, such as anti-obesity drug, anti-cancer drug, etc. The muscle wasting is characterized by a gradual loss of muscle mass or weakness and degeneration of muscles, particularly skeletal or voluntary muscles and cardiac muscles.

More specifically, the term “muscle” refers collectively to sinews, muscles, and tendons. The term “muscle function” refers to the ability of muscle to exert its force when contracted and is intended to include muscle strength, muscular endurance, and agility. The muscle strength means the ability of muscle to exert its maximum contractile force to overcome resistance. The muscle strength is controlled by the liver and are proportional to muscle mass.

And, the term “increasing muscle strength” may refer to increasing muscle strength, muscular endurance, instantaneous muscular power, and the like,

And a reduction in muscle mass due to a reduction or contraction of muscle cells, and may be evaluated through measuring temporary maximum muscle strength, such as grip strength, back muscle strength, arm muscle strength, or leg muscle strength, or muscular endurance that enables the repetition of exercise with a predetermined load.

In an embodiment of the present invention, it was tested whether Akkermansia had an effect of inhibiting muscle strength weakness, by measuring the maximum grip strength of mice after the administration of Akkermansia to the mouse after muscle atrophy is induced by dexamethasone and aged mice, and as a result, it was identified that the muscle strength was increased, muscle weight and muscle fiber size is increased.

The term “myoblast” of the present invention refers to a muscle cell in an undifferentiated state, and the differentiation of myoblasts into skeletal muscle cells forms a muscle tissue, so the differentiation of myoblasts is also referred to as myogenesis. The factors involved in such myoblast differentiation include Mef2, serum response factor (SRF), MyoD, Myf5, Myf6, myogenin, myosin heavy chain, and the like, and the differentiation of myoblasts may be determined by the measurement of expression levels of these factors.

In an embodiment of the present invention, myoblasts were cultured by treating skeletal muscle myoblasts with Akkermansia, and then the mRNA expression levels of myogenin and myosin heavy chain, which are representative factors involved in skeletal muscle differentiation, were measured. As a result, it was identified that the treatment with Akkermansia resulted in significant increases in expression of myogenin and myosin heavy chain. It can therefore be seen that Akkermansia has effects of promoting and improving the differentiation of skeletal muscle myoblasts.

Through the above-described test results, it can be seen that the composition containing as an active ingredient at least one selected from the group consisting of Akkermansia muciniphila cells, a culture thereof and a lysate thereof of the present invention has effects of for increasing muscle strength and/or muscle mass, preventing or treating muscle disease, such as muscle atrophy, sarcopenia, cachexia and muscle wasting.

The contents of the Akkermansia muciniphila cells, the culture thereof and the lysate thereof contained in the composition of the present invention are not limited as long as the composition has effects of for increasing muscle strength and/or muscle mass, preventing or treating muscle disease, such as muscle atrophy, sarcopenia, cachexia and muscle wasting, but these may be contained in a content of 0.0001 wt % to 99.9 wt %, and more specifically 0.01 wt % to 80 wt % relative to the total weight of the final composition.

In an embodiment, the composition of the present invention may be a pharmaceutical composition.

The pharmaceutical composition of the present invention may further contain an appropriate carrier, excipient, or diluent that is commonly used in the preparation of a pharmaceutical composition. As used herein, the term “pharmaceutically acceptable carrier” refers to a carrier or a diluent that does not inhibit biological activity and characteristics of a compound to be administered, while causing no stimulation to an organism.

The carrier usable in the present invention is not particularly limited to the kind thereof, and any carrier may be used as long as the carrier is commonly used in the art and is pharmaceutically acceptable. Non-limiting examples of the carrier may include a saline solution, sterile water, Ringer's solution, buffered physiological saline, an albumin infusion solution, a dextrose solution, a maltodextrin solution, glycerol, ethanol, and the like. These may be used alone or in a mixture of two or more thereof.

In addition, the composition of the present invention may be used by addition of other common additives, such as an antioxidant, a buffer and/or a bacteriostatic agent, as needed, and may be formulated into injectable formulations, such as an aqueous solution, a suspension, and an emulsion, pills, capsules, granules, tablets, and the like, by addition of a diluent, a dispersant, a surfactant, a binder, and/or a lubricant. The pharmaceutical composition of the present invention may be manufactured in various formulations according to whether the desired manner of administration is oral administration or parenteral administration.

Non-limiting examples of the formulation for oral administration may include troches, lozenges, tablets, water-soluble suspensions, oily suspensions, formulated powders, granules, emulsions, hard capsules, soft capsules, syrups, elixirs, or the like.

In order to prepare the composition of the present invention into formulations for oral administration, such as tablets or capsules, the composition may contain: a binder, such as lactose, saccharose, sorbitol, mannitol, starch, amylopectin, cellulose, or gelatin; an excipient, such as dicalcium phosphate; a disintegrant, such as corn starch or sweet potato starch; and a lubricant, such as magnesium stearate, calcium stearate, sodium stearyl fumarate, and polyethylene glycol wax. Furthermore, the composition of the present invention, for a capsule formulation, may further contain a liquid carrier, such as a fatty oil, in addition to the aforementioned materials.

For formulations for parenteral administration, the composition of the present invention may be prepared into, for example, a form for injection, such as subcutaneous injection, intravenous injection, or intramuscular injection; and a suppository injectable form; a form for spraying, such as an aerosol, so as to permit inhalation through a respirator, but are not limited thereto. For the preparation into a formulation for injection, the composition of the present invention may be mixed with a stabilizer or a buffer in water to prepare a solution or a suspension, which is then prepared in a unit dose of an ampoule or vial. When the composition is formulated into a spray, such as an aerosol, a propellant or the like may be mixed with an additive so that a water-dispersed concentrate or a wet powder is dispersed.

The pharmaceutical composition of the present invention may be administered in a pharmaceutically effective amount. The term “pharmaceutically effective amount” of the present invention refers to an amount sufficient for the treatment or prevention of a disease at a reasonable benefit/risk ratio applicable to a medical treatment or prevention, and the level of effective dose may be determined according to: the factors including severity of illness, drug activity, a patient's age, body weight, health, and sex, drug sensitivity of a patient, administration time, administration route, excretion rate, and length of treatment of the composition used in the present invention, and a drug to be mixed or concurrently used in combination with the composition used in the present invention; and other factors well known in the medical field.

The pharmaceutical composition of the present invention may be administered as an individual treatment or in combination with another treatment, and may be administered sequentially or simultaneously with conventional treatments. In addition, the pharmaceutical composition may be administered once or multiple times. The pharmaceutical composition may be administered in an amount at which a maximum effect can be attained with a minimum amount without side effects.

As for the dose of the pharmaceutical composition of the present invention, the pharmaceutical composition of the present invention may be administered to animals including humans at 0.1 mg/kg to 500 mg/kg of body weight per day, but is not limited thereto. The administration frequency of the composition of the present invention may be once or several times using divided doses per day, but is not particularly limited thereto. The above dose is not intended to limit the scope of the present invention in any aspect.

In an embodiment, the composition of the present invention may be a quasi-drug composition.

The term “quasi-drug” of the present invention refers to a product that is not an instrument, machine, or device, among the products used for diagnosing, curing, relieving, treating, preventing, or alleviating diseases of humans or animals, and to a product that is not an instrument, machine, or device, among the products used for exerting pharmaceutical influences on structures and functions of humans or animals, and also encompasses externally applied preparations for skin and personal hygiene products.

In accordance with another aspect of the present invention, there is provided a method for preventing or treating a muscle disease, such as muscle atrophy, sarcopenia, cachexia and muscle wasting, the method including administering, to a non-human subject, a composition containing as an active ingredient at least one selected from the group consisting of Akkermansia muciniphila cells, a culture thereof, a lysate thereof, and an extract of the lysate or culture.

As described above, the Akkermansia muciniphila provided in the present invention has an effect of preventing or treating a muscle disease, such as muscle atrophy, sarcopenia, cachexia and muscle wasting, and thus the composition containing the same can be used to prevent or treating a muscle disease, such as muscle atrophy, sarcopenia, cachexia and muscle wasting.

The “subject” of the present invention may refer to any animal including humans. The animal may be not only a human but also a mammal, such as a cow, a horse, a sheep, a pig, a goat, a camel, an antelope, a dog, or a cat, in need of treatment for a similar symptom to the human. The subject may refer to a non-human subject, but is not limited thereto. The subject may include a subject administered anti-cancer drug such as FOLFOX or anti-obesity drug such as GLP-1 agonists who, as a result of taking such drugs, may suffer from loss of muscle mass and/or muscle strength similar to sarcopenia or cachexia. The anti-cancer drug or anti-obesity drug may be administered concurrently or sequentially with the composition of the present invention.

The “administration” of the present invention refers to an introduction of the composition of the present invention into a subject by any suitable method. The composition of the present invention may be administered through various routes of oral or parenteral administration as long as the composition can reach a target tissue.

As for the administration route of, for example, a pharmaceutical composition, the pharmaceutical composition may be administered through any general route as long as the composition can reach a target tissue. The pharmaceutical composition of the present invention may be administered through a route, such as intraperitoneal administration, intravenous administration, intramuscular administration, subcutaneous administration, intradermal administration, oral administration, intranasal administration, intrapulmonary administration, or rectal administration, according to the desired purpose, but is not particularly limited thereto. However, the composition may be denatured by gastric acid during oral administration, and thus a composition for oral administration needs to be formulated such that an active drug is coated or protected from degradation in the stomach. The composition may also be administered by any device that can deliver an active substance to a target cell.

In accordance with another aspect of the present invention, there is provided a health functional food composition for preventing or treating a muscle disease, such as muscle atrophy, sarcopenia, cachexia and muscle wasting, or increasing muscle strength and/or muscle mass, containing as an active ingredient at least one selected from the group consisting of Akkermansia muciniphila cells, a culture thereof and a lysate thereof.

The health functional food of the present invention may be manufactured by a method that is commonly used in the art, and may be manufactured by adding raw materials and ingredients that are conventionally added in the art. In addition, the health functional food may also be manufactured in any formulation without limitation as long as the formulation is acceptable as a food. The health functional food composition of the present invention may be prepared in various formulations, contains food as a raw material unlike general drugs and thus has an advantage of having no side effects that may occur in long-term use of a drug, can be usually ingested due to excellent portability and thus is very useful, and can be ingested as a supplement for enhancing effects of preventing or treating a muscle disease, such as muscle atrophy, sarcopenia, cachexia and muscle wasting, or increasing muscle strength and/or muscle mass.

The health functional food is not particularly limited in other ingredients, except for the Akkermansia muciniphila cells, the culture thereof, the lysate thereof, and the extract of the lysate or culture as essential ingredients, and may contain several herbal extracts, food supplement additives, or natural carbohydrates as additional ingredients, like in typical health functional foods. The food supplement additives include food supplement additives that are conventional in the art, for example, flavoring agents, savoring agents, coloring agents, fillers, stabilizers, and the like.

Examples of the natural carbohydrates may include ordinary sugars, for example, monosaccharides, such as glucose and fructose; disaccharides, such as maltose and sucrose, and polysaccharides, such as dextrin and cyclodextrin; and sugar alcohols, such as xylitol, sorbitol, and erythritol. In addition to the above-described additives, natural flavoring agents (e.g., rebaudioside A, glycyrrhizin, etc.) and synthetic flavoring agents (saccharin, aspartame, etc.) may be advantageously used as the flavoring agents.

Apart from the above ingredients, the health functional food composition of the present invention may contain various nutrients, vitamins, water (electrolytes), flavoring agents, such as synthetic flavoring agents and natural flavoring agents, coloring agents, extenders (cheese, chocolate, etc.), pectic acid and salts thereof, alginic acid and salts thereof, organic acids, protective colloidal thickeners, pH adjusters, stabilizers, preservatives, glycerin, alcohols, carbonating agents used for carbonated drink, and the like, and may also contain fruit flesh for manufacturing natural fruit juices, fruit juice drinks, and vegetable drinks. These ingredients may be used either alone or in combination. In addition, the health functional food may be in the form of any one of meat, sausage, bread, chocolate, candies, snacks, confectioneries, pizzas, ramen, other noodles, gums, ice creams, soups, drinking water, teas, functional water, drinks, alcoholic drinks, and vitamin complexes.

The health functional food of the present invention may further contain food additives, and the suitability thereof as a “food additive”, unless otherwise specified, is determined by the standards and criteria for the corresponding item in accordance with the General Rules and General Test Methods of the Food Additive Code approved by the Ministry of Food and Drug Safety.

The content of the composition that is added to food including drinks, in the process of manufacturing a health functional food, may be appropriately increased or reduced as needed.

In accordance with another aspect of the present invention, there is provided a feed additive composition for preventing or treating a muscle disease, such as muscle atrophy, sarcopenia, cachexia and muscle wasting, or increasing muscle strength and/or muscle mass, containing as an active ingredient at least one selected from the group consisting of Akkermansia muciniphila cells, a culture thereof and a lysate thereof.

The feed composition may contain a feed additive.

The term “feed additive” in the present invention includes substances that are added to feed for the purpose of various effects, such as supplementing nutrients, preventing weight loss, promoting digestibility of cellulose in the feed, improving milk quality, preventing reproductive disorders, improving a rate of pregnancy, and preventing high-temperature stress during the summer season. The feed additive of the present invention may correspond to supplementary feed according to the Control of Livestock and Fish Feed Act.

The term “feed” in the present invention refers to any natural or artificial diet, a single meal, or the like, or an ingredient of the single meal, which an animal eats, ingests, and digests or which is suitable for eating, ingestion, and digestion. The feed containing the composition according to the present invention as an active ingredient may be prepared in various forms of feed known in the art.

The type of feed is not particularly limited, and feeds commonly used in the art may be used. Non-limiting examples of the feeds may include: vegetable feeds, such as grains, root vegetables, food processing byproducts, algae, fibers, pharmaceutical byproducts, oils and fats, starches, residues, or grain byproducts; and animal feeds, such as proteins, inorganic materials, oils and fats, minerals, oils and fats, single-cell proteins, and animal planktons or foods. These may be used alone or in a mixture of two or more thereof.

The contents of the Akkermansia muciniphila cells, the culture thereof, the lysate thereof, and the extract of the lysate or culture in the feed composition of the present invention may be appropriately adjusted according to the type and age of applied livestock, type of application, and desired effect.

In accordance with another aspect of the present invention, there is provided a pharmaceutical composition for preventing or treating a muscle disease, such as muscle atrophy, sarcopenia, cachexia and muscle wasting, the pharmaceutical composition containing as an active ingredient at least one selected from the group consisting of Akkermansia muciniphila cells, a culture thereof, a lysate thereof, and an extract of the lysate or culture.

In accordance with another aspect of the present invention, there is provided an animal medicine for preventing or treating a muscle disease, such as muscle atrophy, sarcopenia, cachexia and muscle wasting, the animal medicine containing as an active ingredient at least one selected from the group consisting of Akkermansia muciniphila cells, a culture thereof and a lysate thereof. In accordance with another aspect of the present invention, there is provided a method for preventing or treating a muscle disease, such as muscle atrophy, sarcopenia, cachexia and muscle wasting, the method including administering a composition containing as an active ingredient at least one selected from the group consisting of Akkermansia muciniphila cells, a culture thereof and a lysate thereof.

### MODE FOR CARRYING OUT THE INVENTION

Hereinafter, the present disclosure will be described in detail with reference to examples and experimental examples. However, these examples and experimental examples are provided for specifically illustrating the present invention, and the scope of the present invention is not limited to these examples and experimental examples.

### Example 1: Analysis of Myoblast Differentiation Promoting Ability

The Akkermansia muciniphila strain used in the Example 1 and 4 was an Akkermansia muciniphila standard strain (AK; American Type Culture Collection accession number ATCC BAA-835, identical to DSM 22959).

To analyze the effect of Akkermansia strain administration on myoblasts, myoblasts were treated with the live or dead cells of Akkermansia strain to measure the expression levels of myogenic regulatory factors.

Specifically, C2C12 skeletal muscle myoblasts were purchased from ATCC (USA) and cultured in DMEM containing 10% FBS, 100 U/mL penicillin, and 100 μg/mL streptomycin, at 37° C. under a 5% CO₂ condition. For the induction of differentiation of the cells, the cells were dispensed at 5×10⁵ cells/mL in 6-well plates, and when the cells grew to 90% or more, the medium was exchanged with a differentiation medium containing 2% horse serum, and the cells were cultured for 5 days. The live and dead Akkermansia strains were used after dilution to 1×10⁸ cells/mL in PBS. The medium was exchanged every two days, and the differentiation culture was ended after 5 days. Thereafter, the mRNA expression levels of myogenin (Myog) and myosin heavy chain (MyHC) were measured by qRT-PCR.

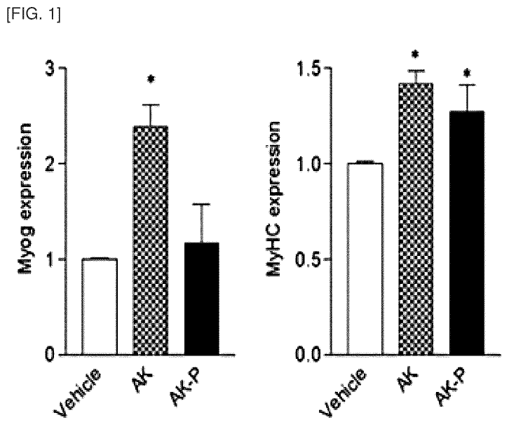

The results identified that the expressions of myogenin and myosin heavy chain were significantly increased in the administration of the live Akkermansia strain (AK group) and dead Akkermansia strain (AK-P group) compared with the vehicle control group (FIG. 1).

It was identified from the results that the Akkermansia strain has an effect of promoting or improving myoblast differentiation.

### Example 2: Analysis of Gene Expression Related Muscle Strength in Muscle Cells

The Akkermansia muciniphila strain used in the Example 2 and 3 was an Akkermansia muciniphila strain (HB05; Korean Culture Center of Microorganisms under accession number KCCM12424P). Akkermansia muciniphila HB05 strain is an obligate anaerobic strain and was confirmed to be safe, lacking genes responsible for pathogenicity, toxicity and antibiotic resistance and antimicrobial substance production. To culture the HB05 strain, a media composition (TABLE 1) was used, and the strain was activated through two successive subcultures over 24-48 hours at 37° C. before fermentation. After fermentation, pasteurization was performed using a low-temperature sterilization method (70° C., 30 minutes), followed by centrifugation to remove the supernatant. The bacterial cells were then mixed with sterile water at a ratio of 2:1 and freeze-dried. After freeze-drying, the sample was ground to produce a powder of pasteurized Akkermansia muciniphila HB05 cells (HB05P) with a dry weight concentration of 2.0×10¹¹ cells/g.

TABLE 1

| Composition | g/L |
|---|---|
| Tryptone | 15 |
| Yeast extract | 10 |
| L-Threonine | 2 |
| D-Lactose | 4.512 |
| N-acetylglucosamine | 1.659 |
| Monopotassium Phosphate | 0.4 |
| Disodium Phosphate | 0.53 |
| Ammonium Chloride | 0.3 |
| Magnesium Chloride | 0.1 |
| Cacium Chloride | 0.11 |
| Sodium Bicarbonate | 4 |
| Vitamin B12 | 0.0001 |
| L-Cystein | 0.5 |

To analyze the effect of Akkermansia strain administration on gene expression related muscle strength, C2C12 skeletal muscle cells were treated with the dead cells of Akkermansia strain to measure the expression levels of Follistatin, Atrogin-1 and MuRF1.

First, mouse skeletal muscle C2C12 cell line (CRL-1772, ATCC, Manassas, VA, USA) was purchased and used. To assess the cytotoxicity of dexamethasone and HB05P on C2C12, a WST-1 assay (Cell Proliferation Reagent WST-1, 5015944001, Roche, Basel, Switzerland) was performed. Myoblasts (C2C12) were dispensed at a density of 2.0×104 cells/well into 96-well plates (Corning® 96-well Clear Polystyrene Microplates, 3598, Corning, Glendale, AZ, USA) and cultured at 37° C. in 5% CO₂ conditions for 24 hours. The cultured cells were then treated with varying concentrations of dexamethasone (0, 250, 500, 750, 1000 μM) and HB05P (0, 1.0×10³, 1.0×10⁴, 1.0×10⁵, 1.0×10⁶, 1.0×10⁷ cells/well). After 24 hours of incubation, cell viability was measured using a VersaMax Microplate Reader (Molecular devices, San Jose, CA, USA).

After 24 hours of treatment, when the cell viability of untreated C2C12 myocytes was represented as 100%, the dexamethasone-treated groups (0, 250, 500, 750, 1000 μM) showed a concentration-dependent decrease in cell viability. Especially, a concentration of 250 μM showed a cell viability of 66.6%. Furthermore, the cell viability in the groups treated with various concentrations of HB05P (0, 1.0×10³, 1.0×10⁴, 1.0×10⁵, 1.0×10⁶, 1.0×10⁷ cells/well) was measured as 100, 100.5, 99.3, 10⁵.1, 102.7, 103.3%, respectively, confirming no toxicity. Therefore, subsequent experiments aimed to evaluate the efficacy of HB05P in inhibiting muscle cell decay was done at a fixed dexamethasone treatment concentration of 250 μM and concurrent treatment with various concentrations of HB05P.

C2C12 cell culture was performed using DMEM supplemented with 10% FBS and 1% antibiotic-antimycotic solution as the growth media and cultured under conditions of 37° C. and 5% CO₂. Upon reaching 80% cell confluency, the media was replaced every two days with DMEM containing 2% horse serum and 1% antibiotic-antimycotic solution, to differentiate the myoblasts into myotubes (differentiation media, DM). On days 5 of differentiation, medium containing 250 μM dexamethasone (D4902, Sigma) and various concentrations of HB05P (0, 1.0×10⁴, 1.0×10⁵, 1.0×10⁶ cells/well) was changed once a day for 2 days.

To measure the change in expression level of muscle-related biomarker by HB05P treatment, cells from each experimental group cultured in 6-well plates were collected, and RNA extraction (TaKaRa MiniBEST Universal RNA Extraction Kit, 9767B, Takara, Kusatsu, Japan) and cDNA synthesis (TaKaRa PrimeScript™ 1 st strand cDNA Synthesis Kit, 6110B, Takara) were performed. The synthesized cDNA (stored at −80° C.) was used as a template for Follistatin, Atrogin-1 and MuRF1 primers (TABLE 2). The qPCR program was an initial step of 50° C. for 2 minutes and 95° C. for 2 minutes, followed by 40 cycles of 95° C. for 15 seconds and 60° C. for 1 minute in qRT-PCR system (StepOne™ Real-Time PCR systems, Applied Biosystems, Waltham, MA, USA). The relative gene expression levels were calculated by the 2^{-ΔΔct} method using GAPDH as an internal control.

TABLE 2

| Gene name | | Sequence (5'→3') |
|---|---|---|
| Atrogin-1 | For. | SEQ ID NO: 1 |
| | Rev. | SEQ ID NO: 2 |
| Follistatin | For. | SEQ ID NO: 3 |
| | Rev. | SEQ ID NO: 4 |
| MuRF1 | For. | SEQ ID NO: 5 |
| | Rev. | SEQ ID NO: 6 |
| GAPDH | For. | SEQ ID NO: 7 |
| | Rev. | SEQ ID NO: 8 |

After simultaneous treatment with dexamethasone 250 μM and various concentrations of HB05P (0, 1.0×10⁴, 1.0×10⁵, 1.0×10⁶, 1.0×10⁷ cells/well), qRT-PCR analysis measured the expression levels of Follistatin, Atrogin-1, MuRF1, MyHC and Mef2c, biomarkers indicating muscle strength-related functionality.

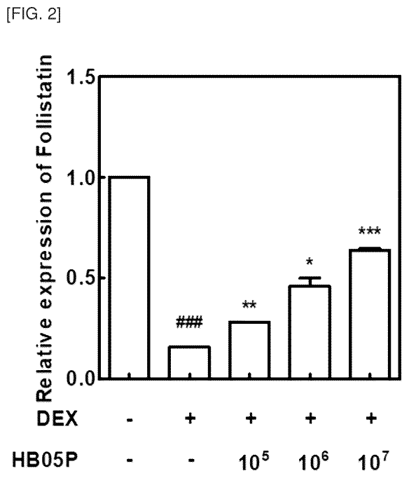

Compared to the group treated with dexamethasone alone, significant increases in Follistatin expression were observed in the groups treated with 1.0×10⁵ and 1.0×10⁶ cells/well of HB05P, by approximately 40.7 and 29.6%, respectively (FIG. 2). Follistatin is known as an antagonist of myostatin which inhibits muscle growth, and it is involved in muscle regeneration and angiogenesis. Moreover, transgenic mice overexpressing Follistatin showed an increase in muscle mass similar to myostatin knockout mice.

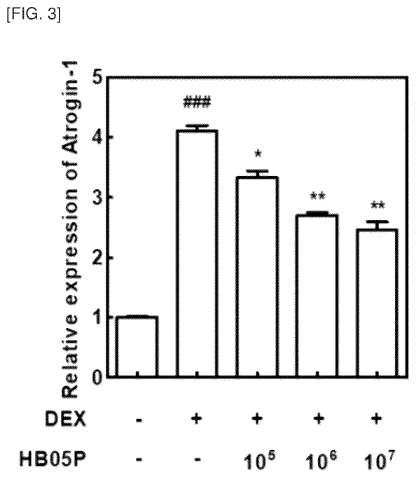

On the other hand, a significant increase in Atrogin-1 expression was observed in the dexamethasone alone treated group compared to the untreated group, and significant decreases in Atrogin-1 expression were noted in the 1.0×10⁵ and 1.0×10⁶ cells/well HB05P-treated groups by approximately 5.5 and 29.9%, respectively, compared to the dexamethasone alone treated group (FIG. 3).

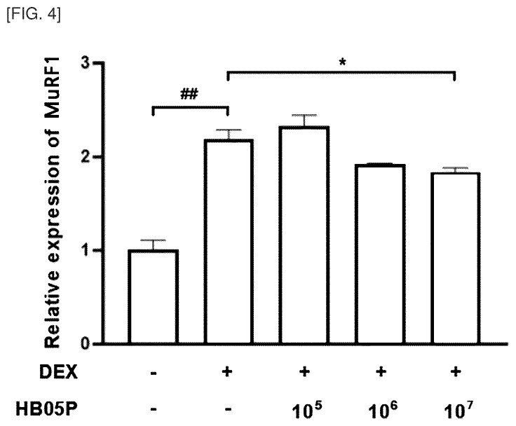

Also, a significant increase in MuRF1 expression was observed in the dexamethasone alone treated group compared to the untreated group, and significant decreases in MuRF1 expression were noted in the 1.0×10⁷ cells/well HB05P-treated groups by approximately 8.3%, compared to the dexamethasone alone treated group (FIG. 4).

Atrogin-1 and MuRF1, a gene related to muscle atrophy, is known to increase in expression due to muscle disuse, nerve damage, inflammation, and other factors. An increase in FBXO32 (Atrogin-1) expression was reported in C2C12 myotubes when compared to the control group in a TNF-α treated C2C12 myotube atrophy model.

Thus, these results shows that oral administration of HB05P was potential in inhibiting muscle cell decay induced by dexamethasone treatment and in improving muscle strength.

### Example 3: Muscle Functions of Dexamethasone-Induced Muscle Atrophy Animal Model in Akkermansia Strain Administration

9-week-old male Sprague Dawley (SD) rats, acclimatized to the laboratory environment for one week. Subsequently, to achieve grip strength values of 1,360-1,400 g, they were divided into five groups with eight rats each and housed in stainless steel bottomed cages, 3-4 rats per cage.

For the group induced with muscle atrophy, dexamethasone was administered intraperitoneally for three weeks to develop a dexamethasone-induced muscle atrophy animal model. The dosage of dexamethasone used was determined to be 1 mg/kg, based on previous research, while the control group (C) received an intraperitoneal injection of PBS during the same period. For sample administration, the untreated group (V) and the muscle-atrophy-control group were orally administered 1 mL of 1×PBS.

The HB05P (2.0×10¹¹ cells/g dry weight) was suspended in PBS to prepare a high concentration sample of 3.0×10⁹ cells/mL PBS, which was then serially diluted to produce medium concentration samples of 3.0×10⁸ cells/mL PBS and low concentration samples of 3.0×10⁷ cells/mL PBS. The prepared high (H), medium (M), and low (L) concentration samples were orally administered daily at 1 mL per day for four weeks, starting one week before the dexamethasone administration and continuing until the end of the experiment.

### 3-1: Analysis of Muscle Strength

To analyze the effect of Akkermansia strain administration on muscle strength, the grip strength test was performed on the animal models.

Body weight and grip strength were measured weekly. The body weight was measured using a Scout™ Pro scale, and grip strength was assessed using a Bioseb grip strength test. The measurement was conducted in grams (g) using a stainless steel T-bar attached to the gauge. The animals were set to grasp the T-bar of the testing apparatus with both forelimbs, and the force exerted to maintain their body weight was recorded.

Compared to the untreated group, the muscle-atrophy-induced groups (muscle-atrophy control, muscle-atrophy+HB05P low, medium, high) showed a significant decrease in body weight over the 3 weeks of dexamethasone treatment (Data not shown). In the 4 weeks of oral administration of the sample, a significant increase in body weight was observed in the muscle-atrophy+HB05P low, medium, high groups compared to the muscle-atrophy control group, indicating a recovery effect due to HB05P administration.

To evaluate the induction of muscle atrophy in experimental animals by dexamethasone, grip strength (muscle strength) of all groups was measured weekly. The results showed that compared to the untreated group, grip strength in the muscle-atrophy control group decreased from the first week after dexamethasone treatment, effectively inducing muscle atrophy. The effect of HB05P intake on muscle strength enhancement was also examined (TABLE 3).

TABLE 3

| | | | Grip strength |
|---|---|---|---|
| | Grip strength (g) | | (units of AUC) |
| Groups | 3 weeks | 4 weeks | 1-4 weeks |
| V | 1,700.73 ± 52.33^{a} | 2,001.62 ± 41.78ª | 5,038.04 ± 92.01ª |
| C | 1,488.77 ± 29.63^{b} | 1,348.19 ± 45.53^{c} | 4,334.61 ± 68.21^{b} |
| L | 1,638.44 ± 36.56^{a} | 1,743.44 ± 43.87^{b} | 4,734.69 ± 79.11^{b} |
| M | 1,711.55 ± 29.12^{a} | 1,746.06 ± 42.71^{b} | 4,827.75 ± 66.35^{ab} |
| H | 1,716.25 ± 37.28^{a} | 1,808.04 ± 62.33^{b} | 4,828.68 ± 83.17^{ab} |

All values are reported as mean ± SEM (n = 8).

In the 3 weeks of HB05P administration, significant increases in grip strength were observed in the muscle-atrophy+HB05P low, medium, high groups compared to the muscle-atrophy control group, reaching levels similar to the untreated group. In the 4 weeks of the administration, the muscle-atrophy+HB05P low, medium, high groups also showed significantly higher grip strength compared to the muscle-atrophy control group.

The change in grip strength from weeks 1-4, represented as the area under the curve (AUC), also showed that the muscle-atrophy control group had significantly decreased muscle strength compared to the untreated group, and the muscle-atrophy+HB05P low, medium, high treatment groups showed significant improvements in muscle strength compared to the muscle-atrophy control group.

These results suggest that HB05P administration helps improve grip strength by aiding in the recovery from weight loss induced by dexamethasone, potentially leading to improvements in fat mass, lean mass, and muscle mass.

### 3-2: Analysis of Body Composition

To investigate the specific changes in body composition due to HB05P administration, DEXA analysis was performed.

After the experiment of 3-1 concluded, before sacrifice, changes in muscle mass were assessed through body composition measurements such as body weight, body fat, and lean mass. These measurements were made using a dual-energy X-ray absorptiometry device (DEXA, iNSiGHT VET DXA, Osteosys Corp., Seoul, Korea).

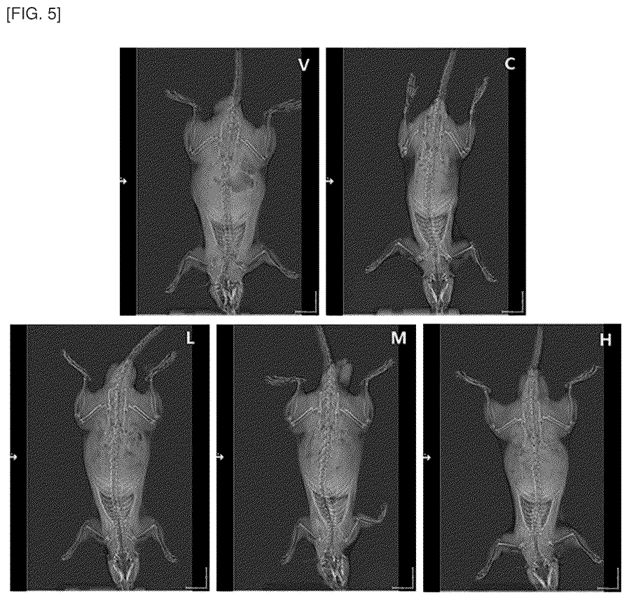

The images of DEXA measurements showed in FIG. 5, and overall improvement in body composition in the HB05P-treated groups showed in TABLE 4.

TABLE 4

| | Body composition | | | | |
|---|---|---|---|---|---|
| Groups | Total BW (g) | Fat (9) | Lean (g) | Lean of hindlimb (g) | Lean of hindlimb/BW (%) |
| V | 387.65 ± 7.40^{a} | 35.39 ± 2.72^{a} | 345.21 ± 5.00^{a} | 55.83 ± 1.86^{a} | 14.40 ± 0.38^{a} |
| C | 274.53 ± 5.09^{b} | 20.11 ± 0.57^{b} | 248.21 ± 4.94^{c} | 33.25 ± 1.32^{c} | 12.12 ± 0.46^{c} |
| L | 292.77 ± 3.70^{b} | 22.15 ± 1.46^{b} | 264.30 ± 2.64^{b} | 36.04 ± 0.85^{bc} | 12.33 ± 0.35^{bc} |
| M | 292.06 ± 6.27^{b} | 20.59 ± 1.00^{b} | 262.28 ± 1.56^{b} | 39.15 ± 1.06^{b} | 13.42 ± 0.30^{ab} |
| H | 291.80 ± 3.08^{b} | 23.40 ± 2.86^{b} | 265.46 ± 5.55^{b} | 39.52 ± 1.37^{b} | 13.55 ± 0.47ª |

All values are reported as mean ± SEM. (n = 8).

Groups induced with muscle-atrophy by dexamethasone (muscle-atrophy control, muscle-atrophy+HB05P low, medium, high) showed a significant decrease in total body weight compared to the untreated group, while the muscle-atrophy+HB05P low, medium, high groups showed a significant increase in total body weight compared to the muscle-atrophy control group.

Additionally, while fat mass significantly decreased in the groups induced with muscle-atrophy by dexamethasone compared to the untreated group, no significant differences in fat mass were observed among the muscle-atrophy control and muscle-atrophy+HB05P low, medium, high groups.

Comparing lean body mass (composed of muscle and minerals, excluding fat mass) with the untreated group, the dexamethasone-treated groups showed a significant decrease in lean body mass, while the muscle-atrophy+HB05P low, medium, high groups showed a significant increase in lean body mass compared to the muscle-atrophy control group. For the lean mass of the hindlimb area, the muscle-atrophy control group treated with dexamethasone showed a significant decrease compared to the untreated group, and the lean mass values for the muscle-atrophy+HB05P low, medium, high groups increased in dose-dependent manner.

When converted to a percentage of body weight, the lean mass of the hindlimb area in the muscle-atrophy control group was about 12% of body weight, whereas in the muscle-atrophy+HB05P medium and high groups, it was over 13%, indicating an increase in lean mass due to HB05P intake.

It should be noted that the experiment measured increase in lean mass of the hindlimb to predict and validate the potential of HB05P for muscle mass increase across skeletal muscle types (GC, TA, and Sol).

### 3-3: Analysis of Muscle Mass

To analyze the effect of Akkermansia strain administration on muscle mass, the muscle mass relative to body weight was measured.

Specifically, the tibialis anterior (TA), gastrocnemius (GC), and soleus muscles (Sol) were isolated from the left and right limbs of each mouse and weighed, and then measured the muscle weight (TABLE 5).

TABLE 5

| | Muscle mass (g) | | |
|---|---|---|---|
| Groups | GC | TA | Sol |
| V | 4.85 ± 0.09^{a} | 2.44 ± 0.12ª | 0.32 ± 0.01^{a} |
| C | 3.22 ± 0.02^{c} | 1.67 ± 0.08^{b} | 0.25 ± 0.01^{c} |
| L | 3.56 ± 0.08^{b} | 1.85 ± 0.05^{b} | 0.30 ± 0.01^{ab} |
| M | 3.66 ± 0.09^{b} | 1.88 ± 0.03^{b} | 0.29 ± 0.01^{ab} |
| H | 3.49 ± 0.08^{b} | 1.88 ± 0.07^{b} | 0.28 ± 0.01^{b} |

All values are reported as mean ± SEM (n = 8).

In a dexamethasone-induced muscle atrophy rat model, it was confirmed that muscle mass increased following HB05P intake.

Compared to the muscle-atrophy control group, a decrease in the weight of the gastrocnemius muscle (GC) was observed in the untreated group, while the gastrocnemius muscle in the muscle-atrophy+HB05P low, medium, high groups significantly increased.

The weight of the tibialis anterior (TA) muscle decreased in the muscle-atrophy control group compared to the untreated group. The weight increased in the muscle-atrophy+HB05P low, medium, high groups compared to the muscle-atrophy control group but the difference was not statistically significant.

The weight of the soleus muscle (Sol) decreased in the muscle-atrophy control group compared to the untreated group, but increased in the muscle-atrophy+HB05P low, medium, high groups with statistical significance.

### 3-4: Identification of Muscle Fiber Amount

To analyze the effect of Akkermansia strain administration on muscle fibers, the muscle fiber volume was measured.

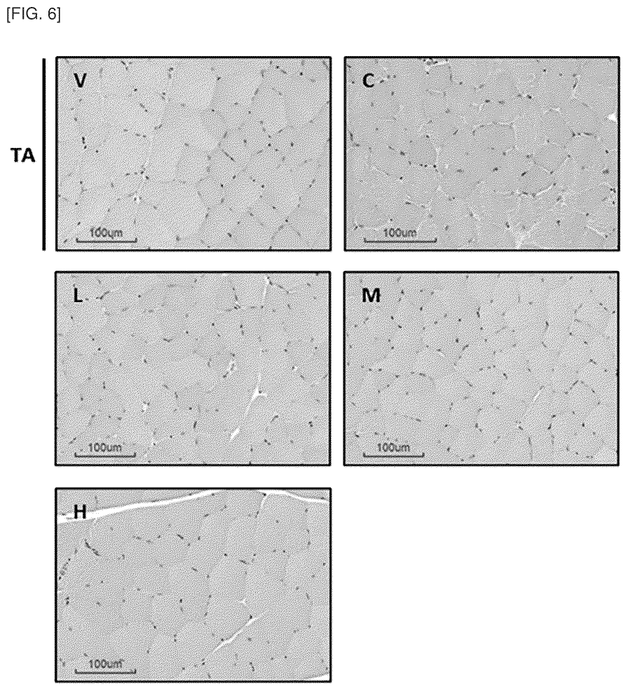

After confirming the significant increase in muscle mass due to HB05P intake, H&E staining was performed to compare the cross-sectional area (CSA) size of muscle fibers (FIG. 6).

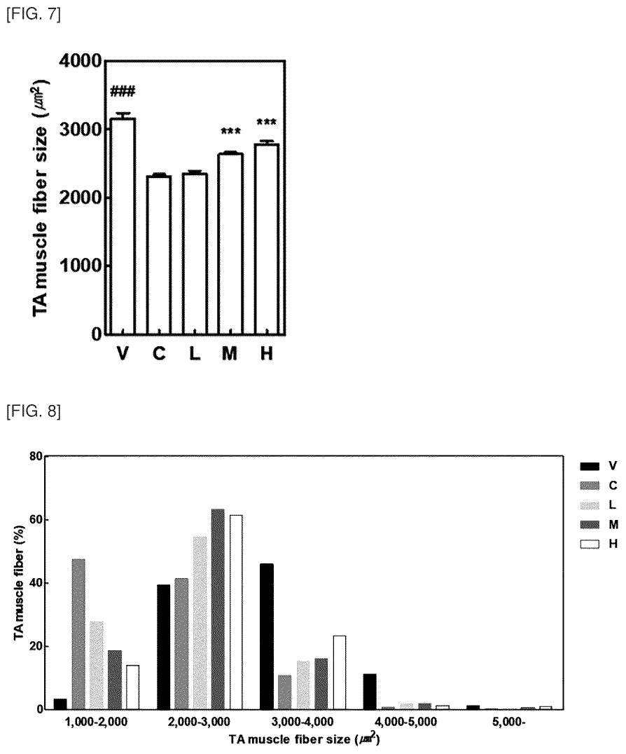

The results (TABLE 6) showed that the cross-sectional area of muscle fibers in the muscle-atrophy control group decreased by about 27% compared to the untreated group, and in the muscle-atrophy+HB05P medium and high groups, the area increased by approximately 14 and 20%, respectively (FIG. 7).

TABLE 6

| | Muscle fiber size (μm²) |
|---|---|
| Groups | TA |
| V | 3,157.17 ± 80.28^{a} |
| C | 2,313.56 ± 30.38^{d} |
| L | 2,349.49 ± 43.54^{d} |
| M | 2,634.59 ± 29.46^{bc} |
| H | 2,775.56 ± 52.23^{b} |

All values are reported as mean ± SEM (n = 8)

There was no significant difference between the muscle-atrophy+HB05P low treatment group and the muscle-atrophy control group. In terms of the percentage of cross-sectional area size ratio (%), the highest proportion was observed in the muscle-atrophy control group within the <2,000 μm² range among untreated group (4.0%), muscle-atrophy control group (39.0%), muscle-atrophy+HB05P low (34.6%), medium (21.2%), and high (19.2%) groups. In the >4,000 μm² range, however, it was observed increases in the cross-sectional area size of TA due to HB05P intake in all groups of untreated group (15.6%), muscle-atrophy+HB05P low (2.4%), medium (5.4%), and high (7.6%) groups compared with muscle-atrophy control group (FIG. 8).

### Example 4: Muscle Functions of Muscle Atrophy Animal Model by Aging in Akkermansia Strain Administration

The 57BL/6 male mice aged 100 weeks were used as animal models for muscle atrophy. The mice were divided into a vehicle group (control group) administered with only BT™ broth used for Akkermansia culture, an AK group in which a live Akkermansia muciniphila strain cultured in BT™ broth was administered at 3×10.sup.8 cells, and an AK-P group in which a dead strain obtained by heating the cultured Akkermansia strain at 70° C. for 30 minutes was administered. The administration for each group was conducted orally once a day for 20 weeks.

### 4-1: Analysis of Muscle Strength

To analyze the effect of Akkermansia strain administration on muscle strength, the grip strength test was performed on the animal models.

Each mouse was allowed to hold a wire mesh, connected to a muscle strength probe of a grip strength meter, with four limbs, and then the tail was carefully pulled backward to measure the maximum grip strength at the moment when the mouse gripped the wire mesh. The grip strength test was performed using the grip strength meter at 8 weeks and 16 weeks of strain administration.

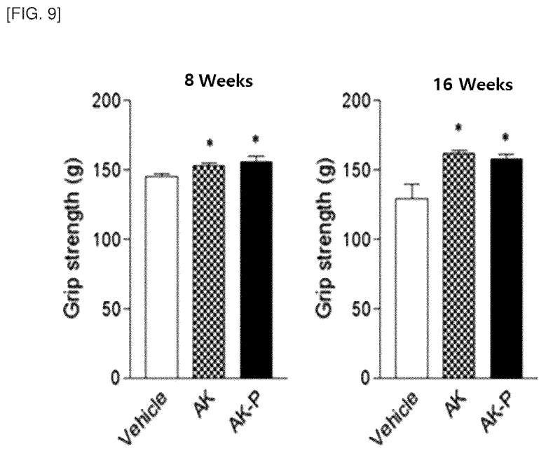

The results identified that at 8 weeks of strain administration, the grip strength of the vehicle control group was 145±2.2 (g), and in comparison, the grip strengths of the live cells of Akkermansia strain administration group (AK group) and the dead cells of Akkermansia strain administration group (AK-P group) were 153±1.5 (g) and 156±4.2 (g), respectively, indicating increases in muscle strength. The results identified that at 16 weeks of strain administration, the grip strength was 130±10 (g) in the vehicle group, indicating a deterioration in muscle strength, but 162±2.0 (g) in the AK group and 157±3.7 (g) in the AK-P group, indicating increases in muscle strength (FIG. 9).

It could therefore be seen from the results that the administration of live or dead cell Akkermansia strain inhibits or ameliorates a decrease of muscle strength.

### 4-2: Analysis of Muscle Mass

To analyze the effect of Akkermansia strain administration on muscle mass, the muscle mass relative to body weight was measured.

Specifically, the tibialis anterior (TA), gastrocnemius (GC), and soleus muscles were isolated from the left and right limbs of each mouse and weighed, and then converted into the muscle weight relative to the body weight (TABLE 7).

TABLE 1

| | TA muscle | GC muscle | Soleus muscle |
|---|---|---|---|
| Control | 3.03 ± 0.08 mg | 7.29 ± 0.14 mg | 0.52 ± 0.04 mg |
| AK group | 3.53 ± 0.07 mg | 8.07 ± 0.19 mg | 0.62 ± 0.01 mg |
| AK-P group | 3.42 ± 0.07 mg | 8.62 ± 0.30 mg | 0.63 ± 0.02 mg |

As a result, the TA muscle weighed 3.03 ± 0.08 mg in the vehicle control group, 3.53 ± 0.07 mg in the live Akkermansia strain administration group (AK group), and 3.42 ± 0.07 mg in the dead Akkermansia strain administration group (AK-P), indicating that the administration of the live Akkermansia strain (AK group) or Akkermansia strain (AK-P) significantly increased muscle mass.

The GC muscle also weighed 7.29±0.14 mg in the vehicle control group, and 8.07±0.19 mg and 8.62±0.30 mg in the live Akkermansia strain administration group (AK group) and the dead Akkermansia strain administration group (AK-P group), respectively, indicating significant increases in muscle mass compared with the vehicle control group.

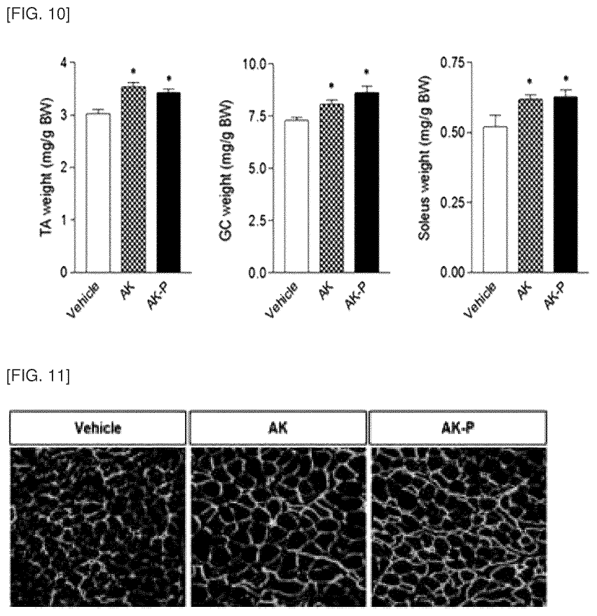

Similarly, the soleus muscle also weighed 0.52±0.04 mg in the vehicle control group, 0.62±0.01 mg in the live Akkermansia strain administration group (AK group), and 0.63±0.02 mg in the dead Akkermansia strain administration group (AK-P), indicating significant increases in muscle mass compared with the vehicle control group (FIG. 10).

Since an increase in muscle mass compared with the control group was shown for each of the three types of muscles, the Akkermansia strain was identified as significantly inhibiting the muscle mass reduction.

### 4-3: Identification of Muscle Fiber Amount

To analyze the effect of Akkermansia strain administration on muscle fibers, the muscle fiber volume was measured.

Specifically, the weight of left tibialis anterior (TA) muscle was measured. Thereafter, the muscle was fixed in 10% formalin and made into frozen sections and subjected to immunostaining for laminin, a main component of the muscle basement membrane. It was identified that strong fluorescence appeared in the Akkermansia strain administration groups (AK and AK-P) (FIG. 11).

Next, confocal microscopy observation and muscle fiber imaging were performed, and the size distribution was obtained for respective cross-sectional sizes of muscle fibers from 500 μm.sup.2 or smaller to 3500 μm.sup.2 or larger by using an image analysis program, and the mean size of all of the muscle fibers was calculated.

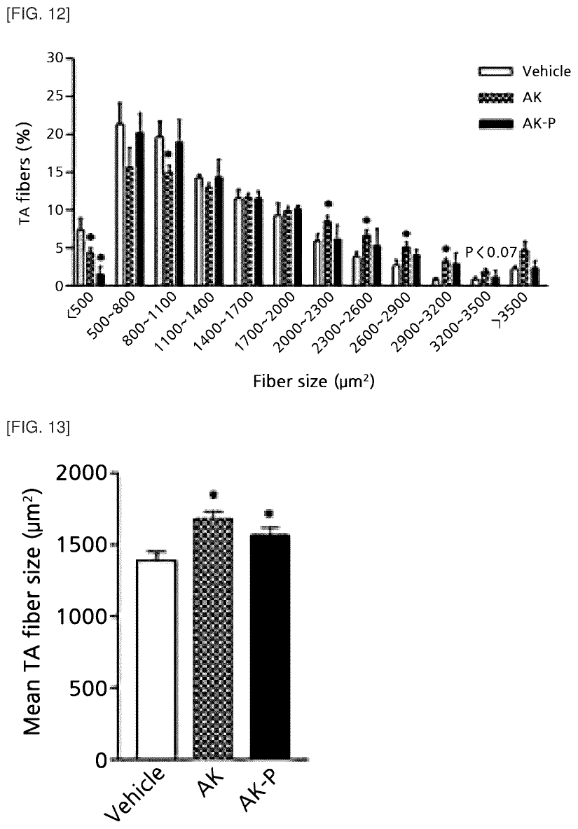

The results identified that live cells of Akkermansia strain administration group (AK group) or dead cells of Akkermansia strain administration group (AK-P group), compared with the vehicle control group, showed significant decreases in the number of small-sized muscle fibers with a TA muscle fiber cross-section size of 500 μm.sup.2 or smaller, but increases in the number of large-sized muscle fibers with a size of 2000 μm.sup.2 or more (FIG. 12).

Also, as a result of calculating the mean size of all of the muscle fibers including from small to large sizes, the mean size was 1,392.5±59.5 μm.sup.2 in the vehicle control group, and 1,681.8±47.7 μm.sup.2 and 1,567.1±50.3 μm.sup.2 in the live Akkermansia strain administration group (AK group) or dead Akkermansia strain administration group (AK-P group), respectively, indicating that the mean size of all of the muscle fibers was significantly increased in the aged mice administered with the live Akkermansia strain (AK group) or dead Akkermansia strain (AK-P group) (FIG. 13).

It was identified from the results that the Akkermansia strain has an effect of inhibiting muscle fiber atrophy.

While the present invention has been described with reference to the particular illustrative embodiments, a person skilled in the art to which the present invention pertains can understand that the present invention may be embodied in other specific forms without departing from the technical spirit or essential characteristics thereof. Therefore, the embodiments described above should be construed as being exemplified and not limiting the present invention. The scope of the present invention is not defined by the detailed description as set forth above but by the accompanying claims of the invention, and it should also be understood that all changes or modifications derived from the definitions and scopes of the claims and their equivalents fall within the scope of the invention.

1. A method for preventing or treating a muscle disease in a subject in need thereof, the method comprising administering to the subject a composition comprising as an active ingredient at least one selected from the group consisting of Akkermansia muciniphila cells, a culture thereof and a lysate thereof.

2. The method of claim 1, wherein the method is increasing myogenesis by increasing mRNA expression levels of Follistatin and/or decreasing muscle atrophy reducing mRNA expression levels of Atrogin-1 in muscle cells.

3. The method of claim 2, wherein Akkermansia muciniphila cells are live cells, dead cells, or a combination thereof.

4. The method of claim 3, wherein Akkermansia muciniphila is cultured in a glucose-free medium, which comprises lactose, N-acetylhexosamine, L-threonine, cobalamin, tryptone and yeast extract.

5. The method of claim 4, wherein Akkermansia muciniphila is dead cells, which are heat-treated and then dried.

6. The method of claim 5, wherein the composition comprises Akkermansia muciniphila dead cells and carrier, wherein a content of Akkermansia muciniphila dead cells in the composition, is 1×10⁵-1×10¹⁵ cell/g.

7. The method of claim 1, wherein the composition is a pharmaceutical composition, a quasi-drug composition, an animal medicine composition, a health functional food composition, or a feed additive composition.

8. The method of claim, 1, wherein the muscle disease is selected from the group consisting of sarcopenia, cachexia, and muscle wasting by weight loss.

9. The method of claim 8, wherein the subject is administered GLP-1 agonists.

10. The method of claim 9, wherein the composition and GLP-1 agonists are administered concurrently or sequentially.

11. A method for increasing muscle strength and/or muscle mass in a subject in need thereof, the method comprising administering to the subject a composition comprising as an active ingredient at least one selected from the group consisting of Akkermansia muciniphila cells, a culture thereof and a lysate thereof.

12. The method of claim 11, wherein the method is increasing myogenesis, increasing mRNA expression levels of Follistatin and/or decreasing muscle atrophy by reducing mRNA expression levels of Atrogin-1 in muscle cells.

13. The method of claim 12, wherein Akkermansia muciniphila cells are live cells, dead cells, or a combination thereof.

14. The method of claim 13, wherein Akkermansia muciniphila is cultured in a glucose-free medium, which comprises lactose, N-acetylhexosamine, L-threonine, cobalamin, tryptone and yeast extract.

15. The method of claim 14, wherein Akkermansia muciniphila is dead cells, which are heat-treated and then dried.

16. The method of claim 15, wherein the composition comprises Akkermansia muciniphila dead cells and carrier, wherein a content of Akkermansia muciniphila dead cells in the composition, is 1×10⁵−1×10¹⁵ cell/g.

17. The method of claim 11, wherein the composition is a pharmaceutical composition, a quasi-drug composition, an animal medicine composition, a health functional food composition, or a feed additive composition.

18. The method of claim 11, wherein the subject is administered GLP-1 agonists.

19. The method of claim 18, wherein the composition and GLP-1 agonists are administered concurrently or sequentially.


SEQUENCE LISTING (8 sequences)

SEQ ID NO: 1 - DNA, 25 bp, synthetic construct
  source 1..25: mol_type = other DNA
ccatcaggagaagtggatctatgtt

SEQ ID NO: 2 - DNA, 20 bp, synthetic construct
  source 1..20: mol_type = other DNA
gcttcccccaaagtgcagta

SEQ ID NO: 3 - DNA, 20 bp, synthetic construct
  source 1..20: mol_type = other DNA
aaaacctaccgcaacgaatg

SEQ ID NO: 4 - DNA, 20 bp, synthetic construct
  source 1..20: mol_type = other DNA
aggcattattggtctgatcc

SEQ ID NO: 5 - DNA, 22 bp, synthetic construct
  source 1..22: mol_type = other DNA
aggactcctgcagagtgaccaa

SEQ ID NO: 6 - DNA, 20 bp, synthetic construct
  source 1..20: mol_type = other DNA
ttctcgtccaggatggcgta

SEQ ID NO: 7 - DNA, 25 bp, synthetic construct
  source 1..25: mol_type = other DNA
acggcaagttcaacggcacagtcaa

SEQ ID NO: 8 - DNA, 23 bp, synthetic construct
  source 1..23: mol_type = other DNA
gctttccagaggggccatccaca